(19) United States
(12) Patent Application Publication
(10) Pub. No.: US 2021/0069189 A1
(43) Pub. Date: Mar. 11, 2021

(54) TREATMENT OF NEURODEGENERATIVE DISEASES

(71) Applicant: BENEVOLENTAI BIO LIMITED, London (GB)

(72) Inventors: Peter Richardson, Haverhill (GB); David Sheppard, Wickhambrook (GB)

(21) Appl. No.: 16/644,849

(22) PCT Filed: Sep. 5, 2018
(86) PCT No.: PCT/GB2018/052516

§ 371 Date: Mar. 5, 2020

(30) Foreign Application Priority Data
Sep. 6, 2017 (GB) 1714316.5

(51) Int. Cl. A61K 31/506; A61K 31/496; A61K 31/49; A61K 47/02

(52) U.S. Cl. CPC A61K 31/506; A61K 47/02; A61K 31/49; A61K 31/496

(57) ABSTRACT

Treatment of Neurodegenerative Diseases Methods for the prevention and treatment of neurodegenerative diseases, in particular motor neuron diseases such as amyotrophic lateral sclerosis (ALS), are described, as well as compositions and combined preparations for use in the methods. The methods comprise inhibiting c-Abl, and inhibiting NFκB activation, in the central nervous system of a subject in need of such prevention or treatment. The compositions comprise an inhibitor of c-Abl, and an inhibitor of NFκB activation.

This invention relates to the treatment of neurodegenerative diseases, in particular motor neuron diseases such as amyotrophic lateral sclerosis, and to compositions or combined preparations for use in the methods.

The motor neuron diseases (MNDs) are a group of progressive neurological disorders that destroy motor neurons, the cells that control essential voluntary muscle activity such as speaking, walking, breathing, and swallowing. Normally, messages from nerve cells in the brain (upper motor neurons) are transmitted to nerve cells in the brain stem and spinal cord (lower motor neurons) and from them to particular muscles. Upper motor neurons direct the lower motor neurons to produce movements such as walking or chewing. Lower motor neurons control movement in the arms, legs, chest, face, throat, and tongue.

When there are disruptions in the signals between the lowest motor neurons and the muscle, the muscles do not work properly; the muscles gradually weaken and may begin to waste away and develop uncontrollable twitching (called fasciculations). When there are disruptions in the signals between the upper motor neurons and the lower motor neurons, the limb muscles develop stiffness (called spasticity), movements become slow and effortful, and tendon reflexes such as knee and ankle jerks become overactive. Over time, the ability to control voluntary movement can be lost.

MNDs are classified according to whether they are inherited (familial) or sporadic, and to whether degeneration affects upper motor neurons, lower motor neurons, or both. In adults, the most common MND is amyotrophic lateral sclerosis (ALS), which affects both upper and lower motor neurons. It has inherited and sporadic forms and can affect the arms, legs, or facial muscles. Primary lateral sclerosis (PLS) is a disease of the upper motor neurons, while progressive muscular atrophy (PMA) affects only lower motor neurons in the spinal cord. In progressive bulbar palsy (PBP), the lowest motor neurons of the brain stem are most affected, causing slurred speech and difficulty chewing and swallowing. There are almost always mildly abnormal signs in the arms and legs.

TABLE 1
Classification of Motor Neuron Diseases

| Type | UMN degeneration | LMN degeneration |
|---|---|---|
| Amyotrophic lateral sclerosis | Yes | |
| (ALS) | | Yes |
| Primary lateral sclerosis | Yes | No |
| (PLS) | | |
| Progressive muscular atrophy | No | Yes |
| (PMA) | | |
| Progressive bulbar palsy | No | Yes, bulbar region |
| (PBP) | | |
| Pseudobulbar palsy | Yes, bulbar region | No |

ALS is a progressive, ultimately fatal disorder that disrupts signals to all voluntary muscles. The terms motor neuron disease and ALS are often used interchangeably. ALS most commonly strikes people between 40 and 60 years of age, but younger and older individuals also can develop the disease. Men are affected more often than women. Familial forms of ALS account for 10 percent or less of cases of ALS, with more than 10 genes identified to date. However, most of the gene mutations discovered account for a very small number of cases. The most common familial forms of ALS in adults are caused by mutations of the superoxide dismutase gene, or SOD1, located on chromosome 21.

There is currently no cure or standard treatment for ALS or the other MNDs. Riluzole (Rilutek®), the only prescribed drug approved by the U.S. Food and Drug Administration to treat ALS, prolongs life by 2-3 months but does not relieve symptoms, and has undesirable side effects such as nausea and fatigue. The drug reduces the body's natural production of the neurotransmitter glutamate, which carries signals to the motor neurons. It is believed that too much glutamate can harm motor neurons and inhibit nerve signaling.

The mammalian central nervous system (CNS) is considered to be immunologically privileged, with relatively few resident immune cells and a highly specific blood-brain barrier (BBB). However, considerable evidence supports the presence of immune and inflammatory abnormalities in neurodegenerative diseases. Neuroinflammation is characterised by the activation and proliferation of microglia (microgliosis), astrogliosis, and infiltrating immune cells. It is a pathological characteristic of many neurodegenerative diseases including Alzheimer's disease (AD), Parkinson's disease (PD), and ALS. Neuroinflammatory responses can be beneficial or harmful to motor neuron survival. These distinct effects are elicited by the different activation states of microglia/macrophages and astrocytes, and are modulated by infiltrating T cells (Zhao et al., J Neuroimmune Pharmacol. 2013; 8(4): 888-899).

Microglia act as the first line of immune defence in the CNS, surveying the surrounding environment through their processes. Microglia are sensitive to pathological changes in the CNS and respond to danger signals from damaged tissue. During the early stage of motor neuron injury in ALS, it is believed that repair signals from motor neurons induce activation of microglial cells to an M2 phenotype. M2 microglia release neuroprotective factors (such as neurotrophic and anti-inflammatory factors) to repair motor neurons and protect against further injury. Astrocytes also participate in the neuroprotective process by secreting neurotrophic factors. As the disease progresses, injured motor neurons release danger signals that transform microglia to a cytotoxic M1 phenotype. M1 microglia release pro-inflammatory cytokines (such as tumor necrosis factor α, TNF-α, and interleukin-1β, IL-1β), and promote neurotoxicity by releasing reactive oxygen species. These pro-inflammatory cytokines further activate microglia leading to excessive neurotoxicity. M1 microglia also promote astrocyte activation. Activated astrocytes acquire deleterious inflammatory phenotypes with release of reactive oxygen species and pro-inflammatory cytokines, which in turn further induce microglial activation and enhance motor neuron degeneration. The activated glial cells also recruit peripheral monocytes/macrophages and T cells into the CNS, which further exacerbate motor neuron degeneration. The neuroinflammatory response in ALS is reviewed by Zhao et al., supra, and by Lewis et al. (Neurology Research International, 2012, Article ID 803701).

Pattern recognition receptors (PRRs) expressed primarily on microglia are the initial responders to tissue insult or damage. PRRs detect unique microbial structures termed pathogen-associated molecular patterns (PAMPs), for example microbial nucleic acids, bacterial secretion systems, and components of the microbial cell wall. Damaged host cells can also trigger PRRs by releasing danger-associated molecular patterns (DAMPs) such as ATP, high-mobility group box 1 (HMGB1), and the heat-shock proteins hsp70 and hsp90. PRRs may either be on the membrane surface, for example Toll-like receptors (TLRs) and C-type Lectin Receptors (CLRs), or inside the cytoplasm, for example Nod-like receptors (NLRs), RIG-I-like receptors (RLRs), and AIM2-like receptors (ALRs). Many PRRs encountering PAMPs and DAMPs trigger signaling cascades that promote gene transcription by nuclear factor-κB (NFκB), activator protein 1 (AP1), and interferon regulatory factors (IRFs). Target genes encode cytokines, interferons, and other proinflammatory or microbicidal proteins.

The Toll-like receptor/Interleukin-1 receptor (TLR/IL-1R) superfamily is a group of structurally homologous proteins characterized by extracellular immunoglobulin-like domains and an intracellular Toll/Interleukin-1R (TIR) domain. The members of the TLR/IL-1R superfamily play a fundamental role in the immune response. These receptors detect microbial components and trigger complex signaling pathways that result in increased expression of multiple inflammatory genes. The superfamily includes the Toll-like receptor (TLR) subfamily, the Interleukin-1 receptor (IL-1R) subfamily, and TIR-domain-containing adaptor proteins (such as MyD88).

A subset of NLRs and ALRs triggers a distinct defence mechanism. These proteins assemble cytosolic protein complexes called inflammasomes. Once active, the inflammasome binds to pro-caspase-1 (the precursor molecule of caspase-1), either via its own caspase activation and recruitment domain (CARD), or via the CARD of the adaptor protein ASC, which it binds to during inflammasome formation. The inflammasome induces autocatalytic cleavage of pro-caspase-1 molecules to form caspase-1, which can carry out a variety of processes in response to the initial inflammatory signal, including the proteolytic cleavage of pro-interleukin (IL)-1β into IL-1β, a pro-inflammatory cytokine.

IL-1β signals through the type I IL-1 receptor/IL-1 accessory protein (IL-1RAcP) complex, leading to NFκB-dependent transcription of pro-inflammatory cytokines (tumor necrosis factor (TNF)-α, IL-6, and interferons) and neutrophil-recruiting chemokines (CXCL1 and CXCL2) in glia. IL-1β induces expression of its own gene (by activating NFκB), which serves as a positive feedback loop that amplifies the IL-1 response.

RNA-binding proteins, and in particular transactive response (TAR) DNA-binding protein 43 (TDP43), are central to the pathogenesis of motor neuron diseases and related neurodegenerative disorders. TDP43 is the major constituent of proteinaceous inclusions that are characteristic of most forms of ALS. TDP43 contains two RNA-recognition motifs (RRMs) involved in RNA and DNA binding, and a glycine-rich carboxy-terminal domain. TDP43 is predominantly nuclear localised. Pathological TDP-43 found in diseased brain and spinal cord is abnormally aggregated, primarily in the cytoplasm. Nearly all sporadic and TDP43 mutant familial cases have TDP43 aggregations (Lee et al., Nat Rev Neurosci. 2011 Nov. 30; 13(1):38-50). The precipitated TDP43 protein is polyphosphorylated, and ubiquitylated. Phosphorylation is tightly associated with aggregation. Acetylation of TDP43 is also part of the aggregation process. Acetylation impairs RNA-binding and promotes accumulation of insoluble, hyper-phosphorylated TDP43 species that largely resemble pathological inclusions in ALS. Acetylation occurs on lysine residues within the RRMs of TDP43. The cytoplasmic histone deacetylase 6 (HDAC6) interacts with TDP43 in vivo. HDAC6 has been shown to deacetylate TDP43, although cytoplasmic TDP43 aggregates were unable to be deacetylated efficiently (Cohen et al., Nat Commun. 2015 Jan. 5; 6:5845).

The SOD1^{G93A} mouse model of ALS is the most widely used animal model for ALS (Gurney et al., 1994, Science 264: 1772-1775). In these mice, a familial mutation in the human SOD1 gene (G93A) that causes ALS is expressed transgenically throughout the body under the control of the endogenous mouse SOD1 promoter. The transgene insertion causes a degenerative disease of lower motor neurons leading to progressive paralysis and eventual death, with the number of transgene copies correlating with severity of disease. Cytoplasmic mislocalization of TDP43 occurs at the end-stage of disease.

The mSOD mouse model recapitulates many aspects of the neuroinflammatory response observed in ALS patients. In the mSOD mouse, increased numbers of activated microglia are observed at early pre-symptomatic stages of disease, and with disease progression to end-stage, microglial numbers in the lumbar spinal cord increase further by nearly 2-fold. Several studies have demonstrated that modulation of the inflammatory response in mSOD mice alters disease progression, leading to suggestions that microgliosis in the mSOD mouse contributed to motoneuron degeneration. However, experiments in which the proinflammatory cytokine TNF-α was ablated in mSOD mice, or where the proliferation of microglia was blocked, had no effect on the rate of disease progression, suggesting that microgliosis does not exacerbate neurodegeneration in the mSOD mouse model.

Some evidence suggests that the epidermal growth factor receptor (EGFR) signaling pathway could play a role in the pathology of neurodegenerative conditions. Treatment with EGFR inhibitors is reportedly neuroprotective in both a rat model of glaucoma (Liu et al., 2006, J Neurosci 26: 7532-7540) and a rat model of spinal cord injury (Erschbamer et al., 2007, J Neurosci 27: 6428-6435). In both studies the authors suggest that EGFR inhibition targets reactive astrocytes. Furthermore, EGFR mRNA expression was found to be upregulated over 10-fold in the spinal cord of human ALS patients as well as in that of the SOD1^{G93A} mouse model (Offen et al., 2009, J Mol Neurosci 38: 85-93), suggesting that pharmacological inhibition of EGFR could be a feasible strategy to slow progression of this disease.

EGFR levels in the spinal cord of SOD1 mice and patients with ALS are increased some 10-fold compared to controls (Offen et al., J Mol Neurosci. 2009 June; 38(2):85-93). The EGFR is strongly implicated in astrocyte activation as a consequence of spinal cord injury (Li et al., Neurochem Int. 2011 June; 58(7):812-9; Li et al., Journal of Neuroinflammation 2014, 11:71). Activated astrocytes express glial fibrillary acid protein (GFAP), inhibit axonal and dendrite elaboration, and release a variety of inflammatory cytokines including TNF, IL-1β and IL-6, which are able to induce neuronal apoptosis (Monje et al., Science. 2003 Dec. 5; 302(5651):1760-5). In addition EGFR inhibitors have been shown to promote neuronal regeneration, while EGF itself can increase astrocyte genesis (Kuhn et al., J Neurosci. 1997 Aug. 1; 17(15):5820-9). Thus, the EGFR can play a pivotal role in astrocyte activation in the spinal cord.

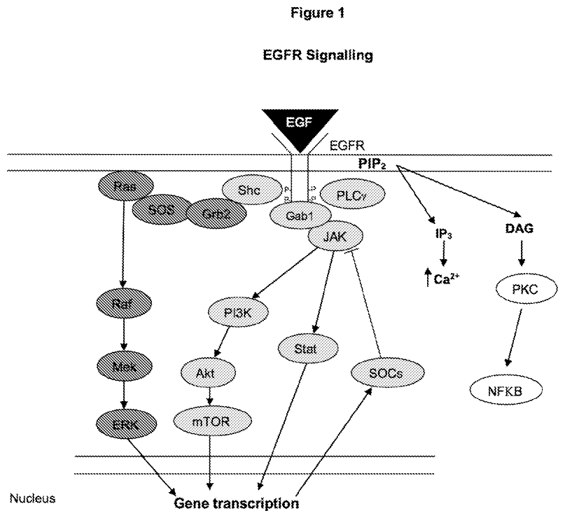

The EGFR signals through multiple pathways, as depicted in FIG. 1, including PI3K/Akt/mTOR, MAPK/ERK, JakSTAT and NFκB. In addition to these pathways, EGFR stimulates c-Abl, a tyrosine kinase, which inhibits EGFR internalisation and autophagy. Internalisation of the receptor results in a reduction of EGFR signaling, thus, blockade of c-Abl reduces EGFR signaling (Tanos and Pendergast, J. Biol. Chem. 2006, 281(43) 32714-23). In addition, the stimulation of autophagy consequent on c-Abl inhibition accelerates the clearance of misfolded proteins, including TDP43.

The EGFR is also involved in microglial activation and proliferation (Qu et al., J Neuroinflammation. 2012 Jul. 23; 9:178), with blockade of the EGFR dramatically reducing the microglial response to LPS. Similarly in vivo, EGFR blockade reduces microglial and astrocyte activation, scar formation and enhanced axonal outgrowth (Qu et al., 2012, supra). These observations suggest that transformation of glial cells in ALS to a MN-toxic phenotype could be significantly modified by blockade of the EGFR. Le Pichon et al., 2013 (PLoS ONE, 8(4): e62342; 1-12) describe a study to test whether erlotinib, an EGFR inhibitor marketed for the treatment of non-small cell lung carcinoma, had a beneficial effect in the SOD1^{G93A} mouse model of ALS. The authors report that erlotinib penetrated into the central nervous system and resulted in a modest yet statistically significant symptom delay as measured by multiple readouts of disease onset and progression. However, the treatment failed to extend lifespan, did not protect motor synapses, and did not correlate with a modulation of markers for astrocytes and microglia. The authors conclude that erlotinib is not efficacious in treating the SOD1 mouse model of ALS. Given the lack of efficacy of erlotinib in this mouse model and the drug's undesirable side effects, which include skin irritation and diarrhea, the authors conclude that erlotinib does not appear to be a good clinical candidate for the treatment of ALS.

There remains, therefore, an urgent need for improved treatment of ALS, and other neurodegenerative diseases.

We have appreciated that effective treatment of neurodegenerative diseases, such as ALS, requires simultaneous correction of multiple dysfunctional pathways and processes in a non-cell autonomous manner.

In a search for alternative methods of treating ALS we have surprisingly found that inhibition of the expression of the stress-activated MAPkinases p38 (MAPK14) and JNK (MAPK8) in ALS patient-derived iAstrocytes protected motor neurons against their toxic effects in vitro. In addition, inhibition of expression of IKKb (the activator of NFκB through phosphorylation of IκB) also protected the motor neurons. This is surprising since Frakes et al., (Neuron, 2015, 81, 1009-1023) reported that, although NFκB signaling has been implicated in ALS (Swarup et al., 2011, J. Exp. Med. 208, 2429-2447), astrocytic NFκB is not been implicated in motor neuron death.

We propose that p38 MAPK-mediated activation of NFκB is involved in ALS iAstrocyte dysfunction. In addition JNK-mediated activation of c-jun and thus, AP-1 mediated inflammation also plays a role in ALS astrocytes.

According to the invention there is provided a method of preventing or treating a neurodegenerative disease, which comprises: inhibiting c-Abl in the central nervous system (CNS) of a subject in need of such prevention or treatment.

There is also provided according to the invention an inhibitor of c-Abl and an inhibitor of MAPK p38/JNK, for use in the prevention or treatment of a neurodegenerative disease.

As used herein, the terms “inhibiting”, “inhibition”, “inhibitor” and the like, may include at least the inhibition of the expression of the indicated proteins; or any inhibition which relates to inhibition of the action of the proteins themselves.

The invention also provides use of an inhibitor of c-Abl and p38MAPK/JNK in the manufacture of a medicament for the prevention or treatment of a neurodegenerative disease.

Within the CNS, c-Abl and MAPK p38/JNK may be inhibited in microglia, astrocytes, or neurons, in microglia and astrocytes, in microglia and neurons, in astrocytes and neurons, or in microglia, astrocytes, and neurons.

The EGFR (also known as ErbB1 or HER1) is a member of the ErbB family of receptors. The other members of the family are ErbB2/HER2/Neu, ErbB3/HER3 and ErbB4/HER4. They are all transmembrane glycoproteins consisting of: (i) a cysteine-rich, extracellular N-terminal ligand binding domain and a dimerization arm; (ii) a hydrophobic transmembrane domain; and (iii) an intracellular, highly conserved, cytoplasmic C-terminal tyrosine kinase domain with several phosphorylation sites. The ectodomain of EGFR has a closed, inactive conformation, and an open, active conformation, which remain in equilibrium with each other. The closed conformation is favoured in the absence of a ligand. Binding of a ligand shifts the equilibrium and stabilizes the open conformation, allowing the dimerization arm to interact with an identical dimerization arm of another receptor molecule to form a homodimer. EGFR also promotes heterodimerization with other members of the HER family, including HER2, HER3 and HER4. Thus, EGFR can initiate cellular signaling cascades by itself, through homodimerzation, or through heterodimerization with other HER family members.

The signaling cascades of the EGFR include the KRAS-BRAF-MEK-ERK pathway, phosphoinositide 3-kinase (PI3K), phospholipase C gamma protein pathway, the anti-apoptotic AKT kinase pathway and the STAT signaling pathway (FIG. 1). The EGFR also activates phospholipase C, which hydrolyses PIP2 to generate Inositol trisphosphate (IP3) and 1,2-Diacylglycerol (DAG). IP3 induces the release of Ca²⁺ from endoplasmic reticulum to activate calcium-regulated pathways. DAG activates the protein kinase C pathway. One of the signaling modules regulated by PKC in the EGFR pathway is the NFκB module. Other signaling modules activated by EGFR include the FAK, JNK, p38 MAPK and ERK5 modules. EGFR induces the JNK pathway through the activation of G proteins such as RAC1 and CDC42, which recruits JNK kinases as well as regulate the actin polymerization.

In some instances EGFR signaling can be inhibited through homologous desensitization mediated by receptor internalization (Yamamoto et al., J Pharmacol. Sci. 2014, 124, 287-93).

In view of the functional involvement of EGFR in various cellular processes, several approaches have been developed that target and interfere with EGFR-mediated effects. Two distinct therapeutic approaches currently employed for targeting EGFR in various human malignancies are the use of small molecule tyrosine kinase inhibitors, and monoclonal antibodies. Tyrosine kinase inhibitors target the intracellular tyrosine kinase domain of EGFR, whereas anti-EGFR antibodies bind to the extracellular domain of EGFR. In addition EGFR signaling can be inhibited through receptor internalization.

Agents that increase EGFR internalization may be used in methods of the invention for the prevention or treatment of neurodegenerative diseases, especially motor neuron diseases.

EGFR internalization is inhibited by c-Abl (Tanos and Pendergast, J. Biol. Chem. 2006, 281(43) 32714-23), so inhibitors of c-Abl increase receptor internalization resulting in abrogation of receptor stimulation by extracellular EGF.

Inhibition of EGFR reduces astrocyte and microglial activation, and inhibits the TLR/IL-1R response to IL-1β and TLR ligands.

In one aspect of the invention, c-Abl is inhibited so as to inhibit phosphorylation of HDAC6 by the EGFR. This may be achieved, for example, by promoting internalization of the EGFR. HDAC6 has been shown to deacetylate TDP43 (Cohen et al., Nat Commun. 2015 Jan. 5; 6:5845). EGFR-mediated phosphorylation of HDAC6 (at Tyr 570) inhibits HDAC6 deacetylase activity (Deribe et al., Sci Signal. 2009 Dec. 22; 2(102):ra84), so blockade of EGFR signaling through internalization should increase HDAC6 activity and thus TDP43 deacetylation, thereby reducing TDP43 aggregation.

In ALS and other neurodegenerative diseases, protein aggregates are frequently found within cells, suggesting a dysfunction in the proteasome or autophagy mechanisms. This may be mediated through the PI3K/AKT/mTOR pathway, which is increased by c-Abl activity (Ertmer, et al., Leukemia 2007 21, 936-942).

In microglia and astrocytes of the mSOD1 mouse, elevated NFκB activity results in associated motoneuron necrosis (Frakes et al., Neuron. 2014 Mar. 5; 81(5):1009-23). In the brain, reduced NFκB activity is associated with reduced post middle cerebral artery occlusion (MCAO) neuronal damage (Vartanian et al., J Neuroinflammation. 2011 Oct. 14; 8:140). In motoneurons necroptosis is mediated through Rip-1, which increases NFκB production, suggesting that targeting NFκB could be a therapeutic option. However, the universal and multi-functional properties of this transcription factor make this difficult.

NFκB activation is also increased by TLR/IL-1R signaling pathways. Upon recognition of their cognate ligands, TLR/IL-1R proteins homo- or hetero dimerize (TLR1/2, TLR2/6, IL-1R/IL-1RacP) and initiate a signaling cascade through recruitment of different combinations of TIR-domain-containing adaptor protein (namely, MyD88, MAL/TIRAP, TRIF, and TRAM) to their TIR domain. All receptors of the superfamily, with the exception of TLR3, use MyD88 to initiate their signaling pathway. In some cases, MyD88 acts in concert with other adaptors, like MAL/TIRAP in the response triggered by stimulation of TLR4, TLR1/2, and TLR2/6. TLR3-mediated signaling requires only the adaptor molecule TRIF, which is also recruited by TLR4 in association with the other adaptor TRAM.

The Applicant has recognised that the increased levels of IL-1β in the spinal cord of SOD1 mice, the ability of mSOD1 to increase IL-16 production, and the fact that such IL-1β accelerates ALS progression and NFκB activation (Meissner et al., Proc Natl Acad Sci USA. 2010 Jul. 20; 107(29):13046-50), suggests that modulation of the TLR4/IL-1 signaling pathway, IL-1β and the innate immune system would be beneficial for the prevention and treatment of neurodegenerative diseases, such as the MNDs, and ALS in particular. In addition, the apparent requirement for active EGFR to generate the LPS response in microglia (Qu et al., Journal of Neuroinflammation (2012) 9, 178) suggests that increased internalization of the EGFR could reduce TLR-stimulated and NFκB mediated inflammation.

The stress activated protein kinases (SAPK) p38 (a.k.a. MAPK11-14) and JNK (a.k.a. MAPK8-10) mediate the production of pro-inflammatory cytokines through increased NFκB activation. They serve to transduce signals from cytokine receptor activation, TLR/IL-1R activation and some small GTP binding proteins such as rac1 (Zarudin and Han, Cell. Res. (2005) 15, 11-18). As such these kinases are central to the pro-inflammatory feedback stimulation of cytokine production mediated by multiple pathways.

P-glycoprotein is a transmembrane drug efflux pump and the most important drug transporter for reducing the entry of drugs into the CNS. In some embodiments, an inhibitor of c-Abl (in particular, a small molecule inhibitor of the c-Abl tyrosine kinase) could be administered with an inhibitor of P-glycoprotein (such as cyclosporine A, ketoconazole, quinidine, ritonavir, verapamil, everolimus, or elacridar (GF120918)) to increase CNS exposure of the inhibitor(s).

According to one aspect of the invention, inhibition of c-Abl signaling is achieved by administering nilotinib, bafetinib or bosutinib to the subject. Nilotinib is a c-Abl tyrosine kinase inhibitor, and also inhibits the kinase activity of c-kit and the p38 and JNK MAPKinases. Bafetinib and bosutinib have similar activity. Thus, administration of one or more of these compounds would reduce c-Abl activity so reducing astrocyte and microglial activation and increasing HDAC6 activity, thereby reducing the acetylation status (and thus inhibiting aggregation) of TDP43.

In addition, nilotinib, bafetinib or bosutinib would inhibit the activation of NFκB through blockade of p38 and JNK MAPkinases and stimulate the removal of misfolded and aggregated TDP43 through the acceleration of autophagy.

Nilotinib is a P-glycoprotein (P-gp) substrate (see Mahon et al., Cancer Res. 2008 December; 68(23): 9809-9816).

Bafetinib is an orally active 2-phenylaminopyrimidine derivative which specifically binds to and inhibits the Bcr/Abl fusion protein tyrosine kinase.

Bosutinib is a synthetic quinolone derivative and dual kinase inhibitor that targets both Abl and Src kinases.

If desired, nilotinib, bafetinib or bosutinib (or a combination of one or more thereof) is co-administered, or administered sequentially with a P-gp inhibitor (such as cyclosporine A, ketoconazole, quinidine, ritonavir, verapamil, everolimus, or elacridar (GF120918)) to increase CNS exposure.

According to the invention there is provided a pharmaceutical composition, which comprises an inhibitor of c-Abl, an inhibitor of NFκB activation and a stimulator of autophagy, with a pharmaceutically acceptable carrier, excipient, or diluent, wherein the inhibitor of c-Abl signaling, and the inhibitor of NFκB activation are different compounds.

According to the invention there is also provided a combined preparation, which comprises: (a) an inhibitor of c-Abl; and (b) an inhibitor of NFκB activation, wherein the inhibitor of c-Abl, and the inhibitor of NFκB activation are different compounds.

There is further provided according to the invention a pharmaceutical composition, or a combined preparation, of the invention, which further comprises a P-glycoprotein inhibitor.

There is also provided according to the invention a composition, which comprises an inhibitor of c-Abl, an inhibitor of NFκB activation, and a P-glycoprotein inhibitor.

There is also provided according to the invention a pharmaceutical composition, which comprises an inhibitor of c-Abl, an inhibitor of NFκB activation, a P-glycoprotein inhibitor, and a pharmaceutically acceptable carrier, excipient, or diluent.

According to the invention there is also provided a combined preparation, which comprises: (a) an inhibitor of c-Abl; (b) an inhibitor of NFκB activation; and (c) a P-glycoprotein inhibitor. The inhibitor of c-Abl, and the NFκB activation may be the same compound, or different compounds. If desired, the inhibitor of c-Abl, and the inhibitor of NFκB activation may be nilotinib, bafetinib or bosutinib. Alternatively, (in particular, where the inhibitor of c-Abl, and the inhibitor of NFκB activation are the same compound) the inhibitor of c-Abl, and the inhibitor of NFκB activation may exclude nilotinib, bafetinib or bosutinib.

The inhibitor of c-Abl, the inhibitor of NFκB activation, and the P-glycoprotein inhibitor may be administered together (i.e. co-administered) or sequentially, in any order. In one preferred aspect, the P-glycoprotein inhibitor is administered before the inhibitor of c-Abl and the inhibitor of NFκB activation.

There is also provided according to the invention a composition, which comprises nilotinib, bafetinib or bosutinib; and a P-glycoprotein inhibitor. The composition may comprise nilotinib, and bafetinib; and a P-glycoprotein inhibitor; or may comprise nilotinib and bosutinib; and a P-glycoprotein inhibitor; or may comprise bafetinib and bosutinib; and a P-glycoprotein inhibitor; or may comprise nilotinib, bafetinib and bosutinib; and a P-glycoprotein inhibitor. The same combinations may also be used mutatis mutandis in the aspects of the invention disclosed below. Nilotinib is a particularly preferred compound for use in all aspects of the present invention.

There is also provided according to the invention a pharmaceutical composition, which comprises nilotinib, bafetinib or bosutinib; and a P-glycoprotein inhibitor, and a pharmaceutically acceptable carrier, excipient, or diluent.

There is further provided a combined preparation, which comprises: (a) nilotinib, bafetinib or bosutinib; and (b) a P-glycoprotein inhibitor.

The P-glycoprotein inhibitor may be selected from the group consisting of cyclosporine A, ketoconazole, quinidine, ritonavir, verapamil, everolimus, or elacridar (GF120918), or quinidine. In particular embodiments, the P-glycoprotein inhibitor is elacridar. Combinations of any two or more of these compounds may also be used.

There is further provided according to the invention a composition, a pharmaceutical composition, or a combined preparation of the invention for use in the prevention or treatment of a neurodegenerative disease.

There is also provided according to the invention use of a composition, a pharmaceutical composition, or a combined preparation of the invention in the manufacture of a medicament for the prevention or treatment of a neurodegenerative disease.

There is also provided according to the invention a method of preventing or treating a neurodegenerative disease, which comprises administering effective amounts of nilotinib, bafetinib or bosutinib; and a P-glycoprotein inhibitor to a subject in need of such prevention or treatment.

The nilotinib, bafetinib or bosutinib and the P-glycoprotein inhibitor may be co-administered, or administered sequentially.

The neurodegenerative disease may be a motor neuron disease, such as amyotrophic lateral sclerosis (ALS).

The neurodegenerative disease may be a familial or sporadic neurodegenerative disease. In a preferred aspect of the invention, the neurodegenerative disease (in particular motor neurone disease, such as ALS) is a familial neurodegenerative disease. In another preferred aspect of the invention, the neurodegenerative disease (in particular motor neurone disease, such as ALS) is a sporadic neurodegenerative disease.

The components of a combined preparation of the invention may be for simultaneous, separate, or sequential use.

The term “combined preparation” as used herein refers to a “kit of parts” in the sense that the combination components (a) and (b) can be dosed independently or by use of different fixed combinations with distinguished amounts of the combination components (a) and (b). The components can be administered simultaneously or one after the other. If the components are administered one after the other, preferably the time interval between administration is chosen such that the effect on the treated disorder or disease in the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a patient sub-population to be treated, or the needs of the single patient, which can be due, for example, to the particular disease, age, sex, or body weight of the patients.

Preferably, there is at least one beneficial effect, for example an enhancing of the effect of one of the components, or a mutual enhancing of the effect of the combination components (a) and (b), for example, a more than additive effect, additional advantageous effects, fewer side effects, less toxicity, or a combined therapeutic effect compared with a non-effective dosage of one or both of the combination components (a) and (b), and very preferably a synergism of the combination components (a) and (b).

In an aspect of the invention, the neurodegenerative disease is a motor neuron disease, such as ALS, PLS, PMA, PBP, or Pseudobulbar palsy, or Alzheimer's disease, or Parkinson's disease, or fronto temporal dementia (FTD).

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

It will be appreciated that, in methods of the invention, the subject should be administered with a therapeutically effective amount of an inhibitor of c-Abl and an inhibitor of a SAPK such as p38 MAPK (and a P-glycoprotein inhibitor, where appropriate).

A “therapeutically effective amount” refers to the amount of an inhibitor of c-Abl and an inhibitor of SAPK that, when administered to a subject for treating a disease, is sufficient to effect such treatment for the disease. The “therapeutically effective amount” will vary depending on the inhibitor(s) used, the disease and its severity and the age, weight, etc., of the subject to be treated.

For example, a therapeutically effective amount of nilotinib, bafetinib or bosutinib is 300-400 mg twice per day when co-administered, or administered sequentially with a P-gp inhibitor. Or a therapeutically effective amount of nilotinib, bafetinib or bosutinib may also be 300-400 mg twice per day when administered without a P-gp inhibitor, for example when administered directly to the CNS (such as directly to the brain or spinal cord).

An inhibitor of c-Abl, and an inhibitor of NFκB activation, may be administered to a subject using any available method and route suitable for drug delivery to the CNS, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery. Where systemic delivery is desired, administration typically involves invasive or systemically absorbed topical or mucosal administration of pharmaceutical preparations. Enteral routes of administration include, but are not necessarily limited to, oral and rectal (e.g., using a suppository) delivery.

Conventional and pharmaceutically acceptable routes of administration include intranasal, intramuscular, intra-tracheal, intrathecal, intracranial, subcutaneous, intradermal, topical, intravenous, intraperitoneal, intra-arterial (for example, via the carotid artery), spinal or brain delivery, rectal, nasal, oral, and other enteral and parenteral routes of administration.

If desired, an inhibitor of c-Abl and/or an inhibitor of NFκB activation is administered by injection and/or delivery, for example, to a site in a brain artery or directly into brain tissue.

In an aspect of the invention, an inhibitor of c-Abl and/or an inhibitor of NFκB activation is administered by direct delivery to the CNS, in particular in to the spinal cord or brain, such as by intracerebroventricular (ICV) administration. Direct administration in to the brain can be undertaken in combination with a controlled delivery device, such as an in-dwelling cannula or pump (for example, implanted subcutaneously at a suitable location). Suitable methods of ICV administration to human subjects are described, for example, in Paul et al., J Clin Invest. 2015; 125(3):1339-1346.

A composition of the invention may be provided in a formulation suitable for, or adapted for, administration directly to the CNS, in particular in to the spinal cord or brain, for example of a human subject. If desired, the formulation comprises one or more electrolytes present in endogenous CSF. In a preferred aspect, the one or more electrolytes are selected from sodium, potassium, calcium, magnesium, phosphorous, and chloride ions. A formulation comprising all of the above electrolytes may be used. In a preferred aspect of the invention, the formulation comprises a solution that closely matches the electrolyte concentrations of endogenous CSF of the subject to be treated, for example a human subject. For example, the formulation may comprise a solution comprising any (or each) of: 100-200 mM sodium ion; 1-5 mM potassium ion; 1-2 mM calcium ion; 0.5-1.5 mM magnesium ion; 0.5-1.5 mM phosphorous ion; and 100-200 mM chloride ion. For example, in one preferred composition of the invention, the formulation comprises a solution comprising 150 mM sodium ion, 3 mM potassium ion, 1.4 mM calcium ion, 0.8 mM magnesium ion, 1.0 mM phosphorous ion, and 155 mM chloride ion.

In one aspect of the invention, a composition of the invention suitable for, or adapted for, administration directly to the CNS, in particular in to the spinal cord or brain, for example of a human subject, does not include a P-glycoprotein inhibitor.

The inhibitor(s) may be administered in a single dose or in multiple doses. A suitable frequency of administration may be at least once per day, every other day, once per week, once every two, three, or four weeks, once every month, two months, or once every three to six months. For example, given its half-life, a suitable frequency of administration of nilotinib is at least twice per day. The inhibitor(s) may be administered over a period of at least a week, at least a month, at least three to six months, at least one, two, three, four, or five years, or over the course of the disease, or the lifetime of the subject.

Where the inhibitor of c-Abl, and the inhibitor of NFκB activation are different compounds, they may be co-administered, or administered sequentially. If the inhibitors are administered sequentially, they may be administered in any order. It will be appreciated that the second inhibitor to be administered should be administered whilst the first inhibitor remains effective. The timing of sequential administration will depend on various factors, such as the respective half-lives of the inhibitors, and their bioavailability. Typically, however, it is expected that the inhibitors should be administered within 96, 72, 48, 36, 24, 12, 6, 5, 4, 3, 2, or 1 hours of each other.

Where a P-gp inhibitor is used, this may be co-administered with the inhibitor(s) of c-Abl and NFκB activation, or the P-gp inhibitor and the inhibitor(s) of c-Abl and NFκB activation may be administered sequentially, for example within 96, 72, 48, 36, 24, 12, 6, 5, 4, 3, 2, or 1 hours of each other (i.e. the P-gp inhibitor may be administered before, or after the inhibitor(s) of c-Abl and NFκB activation). For example, if the inhibitor of c-Abl and NFκB activation is nilotinib, the P-gp inhibitor and the nilotinib may be co-administered, or administered sequentially, for example within 96, 72, 48, 36, 24, 12, 6, 5, 4, 3, 2, or 1 hours of each other. The P-gp inhibitor may be administered before the nilotinib, or the nilotinib may be administered before the P-gp inhibitor. In particular embodiments, the P-gp inhibitor is elacridar.

The amount of P-gp inhibitor that is co-administered, or administered sequentially with the inhibitor of c-Abl and the inhibitor of NFκB activation is likely to depend on the particular inhibitors used. However, a person of ordinary skill in the art can readily determine the appropriate amount of each inhibitor to administer to ensure that a therapeutically effective amount of the inhibitor of c-Abl and the inhibitor of NFκB activation penetrates the CNS of the subject to be treated. For example, based on the results obtained in Example 1 below, it is expected that a therapeutically effective amount of nilotinib, bafetinib or bosutinib for a human subject is 300-400 mg twice per day when co-administered, or administered sequentially with 100 mg per day of the P-gp inhibitor Elacridar.

Methods of preparing pharmaceutical compositions are known, or will be apparent, to those skilled in the art. See, e.g., Remington's Pharmaceutical Sciences, Mack Publishing Company, Easton, Pa., 17th edition, 1985.

Compositions of the invention can be formulated into pharmaceutical compositions by combination with appropriate pharmaceutically acceptable carriers, pharmaceutically acceptable diluents, or pharmaceutically acceptable excipients, and can be formulated into preparations in solid, semi-solid, liquid or gaseous forms, such as tablets, capsules, powders, granules, solutions, injections, inhalants and aerosols.

Pharmaceutically acceptable carriers, excipients, or diluents may include, for example: water, saline, dextrose, glycerol, ethanol, a salt, e.g., NaCl, MgCl₂, KCl, MgSO₄, etc.; a buffering agent, e.g., a phosphate buffer, a citrate buffer, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; glycerol; and the like.

Pharmaceutically acceptable carriers, excipients and diluents are nontoxic to recipients at the dosages and concentrations employed, and can for example include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

For oral preparations, a pharmaceutical composition of the invention may include appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Pharmaceutical compositions for injection can be formulated by dissolving, suspending or emulsifying the active ingredients in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Typically, injectable compositions are prepared as liquid solutions or suspensions; solid forms suitable for solution in, or suspension in, liquid vehicles prior to injection can also be prepared.

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum.

Embodiments of the invention are described below, by way of example only, with reference to the accompanying drawings in which:

FIG. 1 shows a schematic illustration of EGFR signaling;

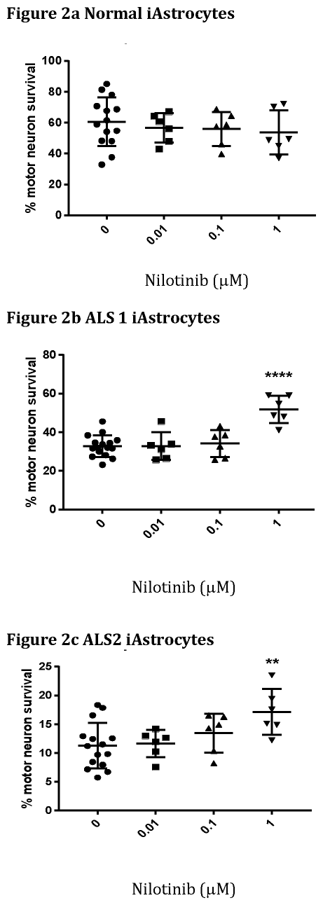

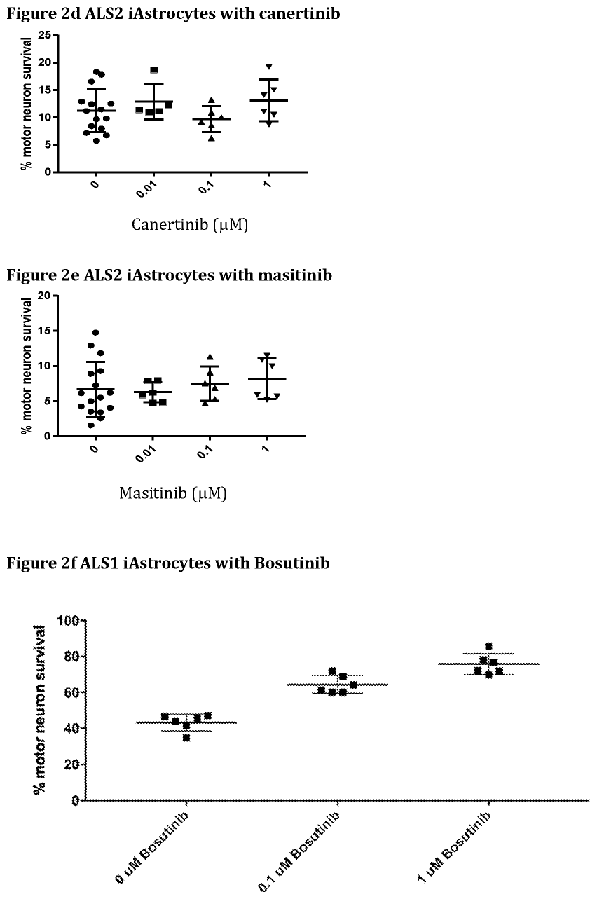

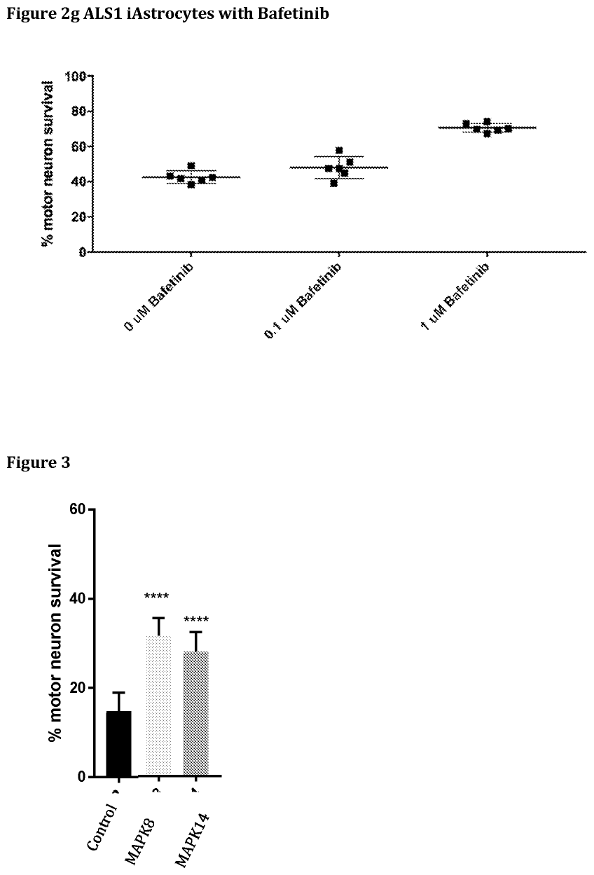

FIG. 2 shows the effect of nilotinib, bafetinib and bosutinib in an in vitro model in which iAstrocytes derived from fibroblasts of two ALS patients (ALS1, and ALS2) were co-cultured with mouse motor neurons. ALS iAstrocytes were pre-treated with various concentrations of nilotinib, bafetinib or bosutinib for 24 hours, prior to murine Hb9-GFP motor neuron seeding in co-culture. The number of viable motor neurons was then measured 24 and 72 hours after motor neuron seeding, and the percentage motor neuron survival was calculated, and then normalised to the untreated control of the respective line. *P<0.05, **P<0.01, ***P<0.001. One-way ANOVA with Dunnett's post-hoc test. Data are mean±SD. n=5-6.

FIG. 3 shows the effect of 5 days treatment of iAstrocytes from patients and controls with shRNA targeting p38MAPK (MAPK14), and JNK (MAPK8) demonstrating that inhibition of the expression of these enzymes protected co-cultured motor neurons. The percentage motor neuron survival was calculated, and then normalised to the untreated control of the respective line. *P<0.05, **P<0.01, ***P<0.001. One-way ANOVA with Dunnett's post-hoc test. Data are mean±SD. n=5-6.

### EXAMPLE 1

### Effect of Nilotinib, Bafetinib and Bosutinib in an In Vitro Model of ALS

This example describes the effect of nilotinib, bafetinib and bosutinib on motor neuron survival in an in vitro model of ALS. This model uses human fibroblast-derived astrocytes and mouse Hb9−GFP+ motor neurons in co-culture (Meyer et al., 2014, PNAS 111, 829-832). The fibroblasts were reprogrammed to induced neural progenitor cells (iNPCs), which were differentiated into Astrocytes. Astrocytes derived from ALS patients cause death of the wild-type Hb9−GFP+ mouse motor neurons in co-culture, a property not seen in astrocytes derived from normal (non-ALS) patients. Interestingly the ALS astrocytes display some abnormalities in metabolism and oxidative stress, which are increased by 10-15 fold in the presence of motor neurons.

Materials and Methods

iNPCs were derived from ALS patient fibroblasts as previously described (Meyer et al. 5 2014, PNAS 111, 829-832), and were differentiated into iAstrocytes by culturing in supplemented DMEM (Sigma) (10% (v/v) FBS (Sigma), 50 units/ml penicillin/streptomycin (Lonza), 1× N-2 supplement (Thermo-Fisher Scientific) for at least 5 days. Murine Hb9− GFP+ motor neurons were differentiated from murine Hb9− GFP+ embryonic stem cells via embryoid bodies, as previously described (Haidet-Phillips et al. 2011, Nature Biotechnology 29, 824-828; Wichterle et al. 2002, Cell 110, 385-397).

3,000 human iAstrocytes were seeded per well on fibronectin-coated 384-well plates. 24 hours later, nilotinib, bafetinib or bosutinib (Cayman Chemical Company, cat. #10010422, #19169 and #12030 respectively) was delivered in 100% drug-grade DMSO to iAstrocyte media using an Echo550 liquid handler (Labcyte). The final concentration of DMSO was 0.24% (v/v) in the media in all wells. Plates were centrifuged at 1,760×g for 60 s. 24 hours later, 2,000 murine Hb9−GFP+ motor neurons were seeded per well in motor neuron media (KnockOut DMEM (45% v/v), F12 medium (45% v/v), KO Serum Replacement (10% v/v), 50 units/ml penicillin/streptomycin (Lonza), 1 mM L-glutamine, 1× N-2 supplement (Thermo-Fisher Scientific), 0.15% filtered glucose, 0.0008% (v/v) 2-mercaptoethanol, 20 ng/ml GDNF, 20 ng/ml BDNF, 20 ng/ml CNTF) and co-cultured on top of the pre-treated iAstrocytes. Plates were centrifuged at 1,760×g for 60 s. Hb9− GFP+ motor neurons were imaged after 24 and 72 hours using an INCELL analyser 2000 (GE Healthcare), and the number of viable motor neurons was counted using the INCELL analyser software (GE Healthcare).

The number of viable motor neurons (defined as GFP+ motor neurons with at least 1 axon) 25 that survived after 72 hours in co-culture was calculated as a percentage of the number of viable motor neurons after 24 hours in co-culture. Percentage survival of motor neurons was then normalised to the DMSO control for each individual iAstrocyte line. One-way ANOVA with Dunnett's post hoc test was performed.

Results

The results, plotted in FIG. 2, show that nilotinib, bafetinib and bosutinib promote a dose-dependent increase in motor neuron survival in co-cultures from three different patients, suggesting that nilotinib, bafetinib and bosutinib will have beneficial effects in patients with ALS. However no such increase in survival was seen with masitinib and canertinib.

Conclusions

It was concluded from these results that the toxic nature of ALS patient-derived astrocytes, as revealed by decreased motor neuron survival in ALS astrocyte/motor neuron co-cultures, is reduced by nilotinib, bafetinib and bosutinib. Inhibition of stress activated kinases also protected the motor neurons. Although not wishing to be bound by theory, these results are consistent with inhibition of c-Abl and p38 and JNK MAPKinases by nilotinib, bafetinib and bosutinib. Such inhibition is expected to inhibit the generation of NFκB, thereby protecting motor neurons.

1. A method of preventing or treating a motor neuron disease, which comprises inhibiting c-Abl, and inhibiting NFκB through inhibition of p38 and/or JNK MAPkinases, in the central nervous system of a subject in need of such prevention or treatment.

2. A method according to claim 1, wherein c-Abl and NFκB activation are inhibited in microglial cells, astrocytes, or neurons of the subject.

3. A method according to claim 1 or 2, wherein c-Abl is inhibited so as to inhibit microglial cell activation and/or formation of inclusions of TDP43.

4. A method according to claim 3, wherein formation of inclusions of TDP43 is inhibited by inducing deacetylation of TDP43.

5. A method according to any preceding claim, wherein c-Abl inhibition reduces EGFR signaling in the subject.

6. A method according to any preceding claim, wherein c-Abl is inhibited by administering an inhibitor of c-Abl to the subject.

7. A method according to claim 6, wherein the c-Abl inhibitor is co-administered, or administered sequentially, to the subject with a P-glycoprotein inhibitor.

8. A method according to any preceding claim, wherein NFκB activation is inhibited so as to inhibit production of IL-1β and NFκB and/or to inhibit formation of inclusions of TDP43.

9. A method according to any preceding claim, wherein NFκB activation is inhibited by inhibiting p38 MARK.

10. A method according to any preceding claim, wherein NFκB activation is inhibited by inhibiting JNK MAPK.

11. A method according to claim 9 or 10, wherein the p38 and JNK MAPkinases are inhibited by administering a small molecule inhibitor to the subject.

12. A method according to claim 9 or 10, wherein the inhibitor is selected from the group consisting of PF-03715455, doramapimod, VX-702, PH-797804, losmapimod, SB203580, PF-03715455, ralimetinib, TAK-715, talmapimod, VX-745, BMS-582949, AZD6703, SB220025, doramapimod, SB202190 and 7-hydroxystaurosporine, lestaurtinib, tamatinib, NVP-TAE684, GSK-1838705A, staurosporine, fedratinib, nilotinib, AST-487, KW-2449, JNJ-28312141, SB203580, and combinations of any two or more of the above.

13. A method according to claim 11 or 12, wherein the inhibitor is nilotinib, bafetinib or bosutinib; or a combination of two or more of said compounds.

14. A method according to any preceding claim, wherein c-Abl, and NFκB activation are inhibited by administering nilotinib, bafetinib or bosutinib, or a combination of two or more of said compounds, to the subject.

15. A method according to any preceding claim, wherein an inhibitor of c-Abl, and an inhibitor of NFκB activation, is administered directly to the brain or spinal cord of the subject.

16. A method according to any of claims 13 and 15, wherein the inhibitor of NFκB activation is co-administered, or administered sequentially to the subject with a P-glycoprotein inhibitor.

17. A method according to any preceding claim, wherein the motor neuron disease is amyotrophic lateral sclerosis (ALS).

18. A method according to any preceding claim, wherein the motor neuron disease is a familial motor neuron disease.

19. An inhibitor of c-Abl, and an inhibitor of NFκB activation, for use in the prevention or treatment of a motor neuron disease.

20. Use of an inhibitor of c-Abl, and an inhibitor of NFκB activation, in the manufacture of a medicament for the prevention or treatment of a motor neuron disease.

21. An inhibitor for use according to claim 19, or use according to claim 20, wherein the c-Abl inhibitor inhibits microglial cell activation and/or formation of inclusions of TDP43.

22. An inhibitor for use according to any one of claim 19 or 21, or use according to any one of claims 20 to 21, wherein the c-Abl inhibitor inhibits formation of inclusions of TDP43 by inducing deacetylation of TDP43.

23. An inhibitor for use according to any one of claim 19, 21, or 22, or use according to any one of claims 20 to 22, wherein the inhibitor of NFκB activation inhibits NFκB activation so as to inhibit production of IL-1β and NFκB and/or to inhibit formation of inclusions of TDP43.

24. An inhibitor for use according to any one of claim 19 or 21, or use according to any one of claims 20 to 21, wherein the inhibitor of NFκB activation inhibits NFκB activation by inhibiting p38MAPK.

25. An inhibitor for use according to any one of claim 19 or 21, or use according to any one of claims 21 to 22, wherein the inhibitor of NFκB activation inhibits NFκB activation by inhibiting JNK MAPK.

26. An inhibitor for use, or use, according to claim 24, wherein the inhibitor of NFκB activation is a small molecule inhibitor of p38MAPK.

27. An inhibitor for use, or use, according to claim 26, wherein the inhibitor of NFκB activation is a small molecule inhibitor of JNK MAPK.

28. An inhibitor for use, or use, according to claim 26 or 27, wherein the inhibitor is selected from the group consisting of PF-03715455, doramapimod, VX-702, PH-797804, losmapimod, SB203580, PF-03715455, ralimetinib, TAK-715, talmapimod, VX-745, BMS-582949, AZD6703, SB220025, doramapimod, SB202190 and 7-hydroxystaurosporine, lestaurtinib, tamatinib, NVP-TAE684, GSK-1838705A, staurosporine, fedratinib, nilotinib, AST-487, KW-2449, JNJ-28312141 and SB203580, and combinations of any two or more of the above.

29. An inhibitor for use, or use, according to claim 23 or 24, wherein the inhibitor is nilotinib, bafetinib or bosutinib; or a combination of two or more of said compounds.

30. An inhibitor for use, or use, according to any of claims 23 to 26, wherein the inhibitor of c-Abl, and of NFκB activation is nilotinib, bafetinib or bosutinib; or a combination of two or more of said compounds.

31. An inhibitor for use, or use, according to any of claims 26 to 30, wherein the inhibitor of c-Abl, and the inhibitor of NFκB activation, are administered directly to the brain or spinal cord.

32. An inhibitor for use, or use, according to any of claims 26 to 30, which further includes a P-glycoprotein inhibitor.

33. An inhibitor for use, or use, according to any of claims 26 to 31, wherein the motor neuron disease is ALS.

34. An inhibitor for use, or use, according to any of claims 26 to 33, wherein the motor neuron disease is a familial motor neuron disease.

35. A pharmaceutical composition, which comprises an inhibitor of c-Abl, and an inhibitor of NFκB activation, and a pharmaceutically acceptable carrier, excipient, or diluent, wherein the inhibitor of c-AbI, and the inhibitor of NFκB activation are different compounds.

36. A combined preparation, which comprises: (a) an inhibitor of c-Abl, and (b) an inhibitor of NFκB activation, wherein the inhibitor of c-Abl, and the inhibitor of NFκB activation are different compounds.

37. A pharmaceutical composition according to claim 35, or a combined preparation according to claim 38, which further comprises a P-glycoprotein inhibitor.

38. A method according to any one of claims 7 to 18, or a pharmaceutical composition according to claim 35 or 37, or a combined preparation according to claim 36, wherein the P-glycoprotein inhibitor is selected from the group consisting of cyclosporine A, ketoconazole, quinidine, ritonavir, verapamil, everolimus, and elacridar, and combinations of any two or more of the above.

39. A composition, which comprises nilotinib, bafetinib or bosutinib, or a combination of two or more of said compounds; and a P-glycoprotein inhibitor.

40. A pharmaceutical composition, which comprises nilotinib, bafetinib or bosutinib, or a combination of two or more of said compounds; and a P-glycoprotein inhibitor, and a pharmaceutically acceptable carrier, excipient, or diluent.

41. A combined preparation, which comprises: (a) nilotinib, bafetinib or bosutinib, or a combination of two or more of said compounds; and (b) a P-glycoprotein inhibitor.

42. A composition according to claim 39, a pharmaceutical composition according to claim 40, or a combined preparation according to claim 41, wherein the P-glycoprotein inhibitor is selected from the group consisting of cyclosporine A, ketoconazole, quinidine, ritonavir, verapamil, everolimus, and elacridar, and combinations of any two or more of the above.

43. A composition according to claim 43, a pharmaceutical composition according to claim 42, or a combined preparation according to claim 41, wherein the P-glycoprotein inhibitor is quinidine.

44. A pharmaceutical composition, which comprises an inhibitor of c-Abl, and an inhibitor of NFκB activation, and a pharmaceutically acceptable carrier, excipient, or diluent, wherein the pharmaceutical composition is suitable for, or adapted for, administration directly to the CNS.

45. A pharmaceutical composition according to claim 44, which comprises one or more electrolytes present in endogenous CSF, wherein the one or more electrolytes is selected from sodium, potassium, calcium, magnesium, phosphorous, and chloride ions.

46. A pharmaceutical composition according to claim 45, which comprises a solution comprising 150 mM sodium ion, 3 mM potassium ion, 1.4 mM calcium ion, 0.8 mM magnesium ion, 1.0 mM phosphorous ion, and 155 mM chloride ion.

47. A pharmaceutical composition according to any of claims 44 to 46, wherein the inhibitor of c-Abl, and the inhibitor of NFκB activation is nilotinib, bafetinib or bosutinib; or a combination of two or more of said compounds.

48. A composition, a pharmaceutical composition, or a combined preparation according to any of claims 39 to 47 for use in the prevention or treatment of a motor neuron disease.

49. Use of a composition, a pharmaceutical composition, or a combined preparation according to any of claims 35 to 47 in the manufacture of a medicament for the prevention or treatment of a motor neuron disease.

50. Use according to any of claims 35 to 49, wherein the motor neuron disease is amyotrophic lateral sclerosis (ALS).

51. Use according to any of claims 48 to 50, wherein the motor neuron disease is a familial motor neuron disease.
